(19) United States
(12) Patent Application Publication
(10) Pub. No.: US 2004/0117005 A1
(43) Pub. Date: Jun. 17, 2004

(54) Stent with drug-delivery system

(76) Inventors: Badari Narayan Nagarada Gadde, Andhra Pradesh (IN); Soma Raju Boopathi Raju, Andhra Pradesh (IN); Reddy Nallamala Krshna, Andhra Pradesh (IN); Raju Penumatsa Raghaya, Andhra Pradesh (IN); K. K. Haridas, Kerala (IN)

Correspondence Address: Ladas & Parry, 26 West 61st Street, New York, NY, 10023, US

(21) Appl. No.: 10/450,576

(22) PCT Filed: Dec. 15, 2000
(86) PCT No.: PCT/IN00/00126

(51) Int. Cl. A61F 2/06

(52) U.S. Cl. 623/1.42; 623/1.15

(57) ABSTRACT

A unique design of the stent that incorporates a drug-reservoir running along the stent struts. One or more drugs can be incorporated within the reservoir. Additionally other drugs can be coated on the surface of the strut enclosing the reservoir drug to facilitate sequential release of drugs. Thus, the design incorporating sequential release of multiple drugs facilitates to tackle the sequential complex biologic processes involved in renarrowing following stent implantation. The design also ensures that the drug present in the reservoir is released exclusively into the adjacent tissue without getting washed away into the blood flowing within the lumen.

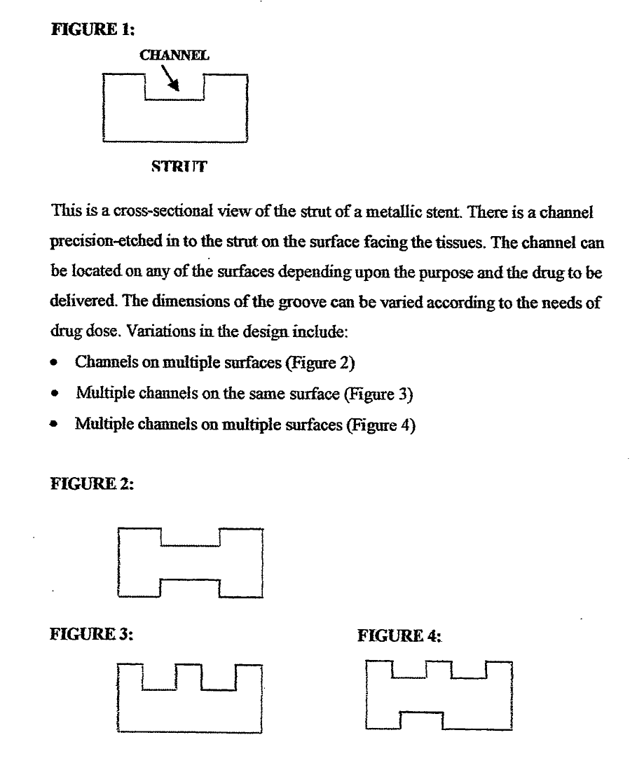

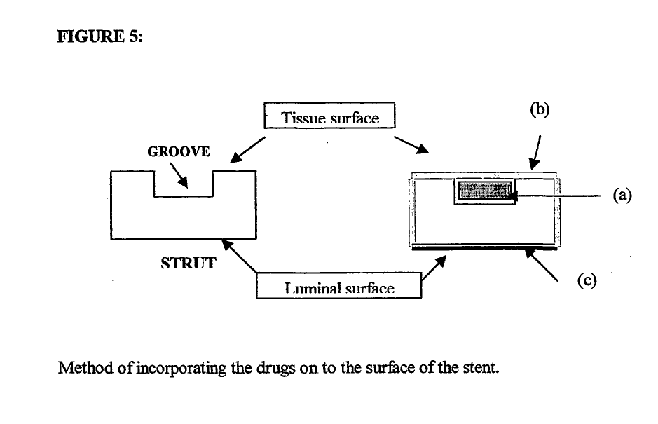

### BACKGROUND OF THE INVENTION

The present invention relates generally to methods of local drug delivery using either stents or balloons. Specifically it relates to unique design of drug coating of stent, which offers sequential release of drugs specifically towards tissue interface.

Before the advent of balloon angioplasty, the method of treatment for blocks in coronary arteries used to be bypass graft surgery. Major limitations of balloon angioplasty, namely abrupt occlusion and late renarrowing, have been overcome to some extent by the development of stents, which act as scaffolding devices. However, renarrowing occurs in 20% to 30% of stents within 6 months. Biological process of renarrowing involves initial platelet aggregation with thrombus, followed by inflammatory response to injury leading to elaboration of various growth factors that stimulate profuse proliferation of cells of inner layer.

Various methods are being adopted to decrease or eliminate the process of renarrowing following stent implantation. These include radiation therapy, either from a luminal source or by means of radiation emitting stents; systemic drugs; biodegradable stents; and coated stents.

Current drug delivery systems are either based on balloon delivery techniques or through coating of stents. Coating of the stents is being evaluated recently Present methods of stent coating are mostly with only one drug, which may not address all the major biological events involved in the process of renarrowing; they do not have full control on luminal washout due to blood flow. The level of drug reaching tissues directly is not predictable Additionally, surface coating of the drug increases the strut thickness, which may affect the expansion properties and radial strength of the stent

### OBJECTS OF THE INVENTION

1. An object of the present invention is to provide a unique design of stent to create a reservoir of drug

2. Additional object includes release of drug specifically into the tissues

3. Another object of the present invention is to provide a design to coat multiple drugs.

4. An additional object of the present invention is to provide a method of sequential drug delivery according to the sequence of biological events.

5. A more particular object of the present invention is to provide a system that can potentially eliminate the risk of renarrowing following stent placement in the vascular structures.

6. Yet another object of the present invention is to use the unique design for local drug delivery, as applicable in malignant tumors

7. These and other objects of the present invention will be apparent from the drawings and detailed descriptions herein

### DESCRIPTION OF THE INVENTION

The present invention is directed to be a unique drug-delivery system designed to release drugs in a sequential fashion.

The device has a running channel in the strut to provide a reservoir for the drug or multiple drugs(a). The channel may run through entire length of stent struts or be limited to certain length of struts avoiding the connecting struts. Either single or multiple drugs may be incorporated in the channel. Timed-release of the drug from the reservoir can be affected by a surface dissolvable coat (b). This coat may be incorporated with another drug that needs to be released before the drug present in the reservoir channel. Both the drugs are released only in to the tissues without getting directly exposed to blood. The luminal surface of the strut can be coated with a different drug (c).

The design offers a unique facility of sequential drug delivery. Luminal surface, which is predominantly exposed to the blood will be coated with any of the anti-thrombotic drugs (heparin, Hirudin, Hirulog, abciximab, synthetic Gp IIb/IIIa blockers) or a natural membrane constituent (phosphotidyl choline). The drug/agent in the groove can have a delayed delivery linked to the dissolution of the surface coat that faces the tissue. The sequential drug delivery is specifically aimed at biological events that occur following a stent implantation. These include in sequence: platelet aggregation and thrombosis, inflammatory reaction and neointimal cell proliferation. Hence, the luminal coating targets the initial event. The coating on the tissue surface can incorporate an anti-inflammatory drug targeting the intermediate event. Finally, the drug in the reservoir can be an anti-cell proliferative agent targeting the main event in the process of tissue response to injury that underlies the phenomenon of restenosis following stent implantation.

Variations can be offered in the choice of drugs, number of drugs, sequence of drug release, and duration of drug release. Variations also include in the strut thickness, the depth of the groove, and running or interrupted groove.

1. A unique design of a stent with a channel running along the length of the struts for incorporating drug or drugs

2. A stent design as claimed in claim 1, which has the channel running on the surface of the strut facing the tissue

3. A stent design as claimed in claim 1, wherein more than one channel be present on the surface

4. A stent design as claimed in claim 2, wherein more than one channel can present on the surface of the strut facing the tissue.

5. A stent design as claimed in claims 1,2,3,&4, wherein a porous surface coating is applied over the surface of the strut containing the channel to control the release of the drug located within the channel

6. A stent design as claimed in claims 1,2,3&4, wherein a biodegradable material coating is applied over the surface of the strut containing the channel to effect delayed release of the drug present in the channel

7. A stent design as claimed in claims 1,2,3&4, wherein the surface coating may incorporate another drug which needs release earlier than the drug present in the channel

8. A stent design as claimed in claim 7, wherein another drug can be coated on to the luminal surface of the strut facing the blood stream

9. A concept wherein sequential local drug delivery to target sequential biological events that occur in response to stent implantation

10. A concept as claimed in claim 9, wherein the coating on the luminal surface may incorporate a drug that specifically inhibits the process of clotting, which is the initial response in the sequence of events. The candidate drugs include platelet inhibitors, heparin and direct thrombin inhibitors.
